(19) United States
(12) Patent Application Publication
(10) Pub. No.: US 2026/0224496 A1
(43) Pub. Date: Aug. 6, 2026

(54) HIGHLY POROUS GAS-BLOWN HYDROGELS FOR DIRECT CELL ENCAPSULATION WITH HIGH CELL VIABILITY

(71) Applicants: Mary Beth Monroe, Syracuse, NY (US); Henry T Beaman, Somerville, MA (US)

(72) Inventors: Mary Beth Monroe, Syracuse, NY (US); Henry T Beaman, Somerville, MA (US)

(73) Assignee: Syracuse University, Syracuse, NY (US)

(21) Appl. No.: 19/153,239

(22) PCT Filed: Feb. 2, 2024
(86) PCT No.: PCT/US2024/014152

§ 371 Date: Aug. 1, 2025

Related U.S. Application Data
(60) Provisional application No. 63/482,884, filed on Feb. 2, 2023.

(51) Int. Cl. A61K 9/48; A61K 9/12; A61K 35/00; A61K 47/32; A61K 47/34

(52) U.S. Cl. CPC A61K 9/4833; A61K 9/122; A61K 9/4825; A61K 9/4866; A61K 35/00; A61K 47/32; A61K 47/34

(57) ABSTRACT

A highly cytocompatible direct cell encapsulation method using rapidly fabricated porous hydrogels. Using sodium bicarbonate and citric acid as blowing agents, photo-curable polymers were used to produce highly porous materials within a matter of minutes. Cells were directly encapsulated within methacrylated poly(vinyl alcohol), gelatin, and poly(ethylene glycol) hydrogels at viabilities as high as 93% by controlling solution variables, such as citric acid content, viscosity, pH, and curing time. Cell viability within the resulting porous constructs was high (>80%) over 14 days of analysis with multiple cell types, thereby providing a simple, versatile, and tunable method for cell encapsulation within highly porous constructs that can be used for the delivery of cell-based therapies.

### CROSS-REFERENCE TO RELATED APPLICATION

The present application claims priority to U.S. Provisional Application No. 63/482,884, filed on Feb. 2, 2023.

### BACKGROUND OF THE INVENTION

### 1. Field of the Invention

The present disclosure relates to cell transplant therapies and, more specifically, to a hydrogel foam that can encapsulate cells without compromising cell viability.

### 2. Description of the Related Art

Cell transplant therapies show potential as treatments for a large number of diseases. The encapsulation of cells within hydrogels is often used to mimic the extracellular matrix and protect cells from the immune response. However, cell encapsulation can be limited due to poor nutrient and waste transport throughout the bulk of larger volume hydrogels. Strategies to address this issue include creating pre-vascularized or porous structured materials. For example, cell-laden hydrogels can be formed by porogen leaching or 3d printing but these techniques involve the use of multiple materials, long preparation times, and/or specialized equipment. Post-fabrication cell seeding in porous scaffolds can result in inconsistent cell density throughout scaffold volumes and therefore typically requires a bioreactor to ensure even cell distribution.

More specifically, a major goal of tissue engineering is to successfully mimic the extracellular matrix (ECM) so that cells can grow and regenerate new, healthy tissue. To that end, many advances in the development of biomaterials with properties such as cell attachment, electroconductivity, and environmental-response, have been engineered. Due to the large water content and soft nature of natural ECM, hydrogels are often used to recapitulate these environments. Hydrogels use a range of hydrophilic polymers, from completely synthetic materials, like poly(vinyl alcohol) (PVA) and poly(ethylene glycol) (PEG), to natural polymers, such as gelatin and fibrin. Often, cell interactions are characterized on flat biomaterial surfaces in vitro. However, cells attached to 2-dimensional constructs do not provide a realistic recreation of the natural ECM environment with cells embedded within 3-dimensional tissues.

To enhance complexity, cells can be encapsulated within hydrogels. In general, cell-encapsulated hydrogels are limited in size due to poor nutrient and waste transport to the center of bulk (solid) scaffolds. Cell-laden hydrogels can be constructed at thicknesses of no greater than 2 mm before issues with diffusion-based nutrient/waste transport are observed. To combat the issue of poor nutrient and waste transport, materials have been engineered to contain either porous or pre-vascularized structures. These materials can be created using methods such as 3D printing, particulate leaching, and gas foaming. Use of 3D printing techniques, such as stereolithography, freeform reversible embedding of suspended hydrogels (FRESH), and two-photon polymerization, has been used to create vascular structures in hydrogels. These methods are often only employed for small construct fabrication, due to the high resolution needed to make vascularized hydrogels where cells are only 100-200 μm away from a fabricated blood vessel. To address these limitations, advancements have been made that allow for rapid printing of high-resolution vascular networks using a specialized form of multi-photon printing called holographic 3D printing. While these systems are promising, their high costs (e.g., Holograph-X by CELLINK costs over $1 million) limits their expanded use and overall translation.

By using highly porous hydrogel materials for cell encapsulation, low-cost scaffolds can be rapidly fabricated. Recently, particulate leached foams were developed using cytocompatible macroscopic porogens made from gelatin and PEG. By changing the amount and size of the porogen used to fabricate the hydrogels, properties such as porosity and pore size can be easily manipulated. The limitation of this method is the additional and tedious fabrication step to make the porogen. Also, particulate leached foams can suffer from poor interconnectivity between pores when fabricated with porogen concentrations <70%, which can limit mechanical property tuning and/or the potential ingrowth of vasculature after transplantation.

An alternative option is the use of gas-blown foams. Gas blowing has been used to create porous biomaterial foams for tissue-engineered scaffolds, traumatic wounds, and aneurysm treatments. Gas-blown foams are produced by chemical blowing agents that release gases, such as CO₂, and/or the physical addition of gases. Gas bubbles get trapped as a polymer is crosslinked to create a porous scaffold. To use gas blowing for cell encapsulation, all foaming components must be cytocompatible. Previous work attempted to encapsulate cells during gas blowing by introducing air bubbles into a gelatin methacrylate (GelMA) solution while UV crosslinking. These foams showed improved cell viability over 7 days in comparison to the non-porous hydrogel controls, but a prolonged procedure involving multiple curing and cell addition steps was needed to successfully produce the constructs. Other work was completed by using sodium bicarbonate (NaHCO₃) and citric acid as chemical blowing agents during cell encapsulation. The reaction between citric acid and NaHCO₃ produces CO₂ as a byproduct. Improved cell survival was seen in porous PEG dimethacrylate (PEGDMA) hydrogels compared to the non-porous controls at 7 days. The major disadvantage of this method was that 50-70% of cells died during the encapsulation process due to the rapid pH changes, high concentrations of citric acid and NaHCO₃, and the cytotoxic crosslinking method. NaHCO₃ is a common component in cell culture media, making it a good candidate to use for gas blowing; however, the authors used NaHCO₃ at concentrations >50 times the amount used in standard culture media (e.g., Dulbecco's Modified Eagle Medium (DMEM)).

### BRIEF SUMMARY OF THE INVENTION

The present invention provides a highly cytocompatible direct cell encapsulation method while rapidly fabricating porous hydrogels. Using sodium bicarbonate and citric acid as blowing agents, photo-curable polymers were employed to produce highly porous materials within a matter of minutes. Cells were directly encapsulated within methacrylated poly(vinyl alcohol), gelatin, and poly(ethylene glycol) hydrogels at viabilities as high as 93% by controlling solution variables, such as citric acid content, viscosity, pH, and curing time. Cell viability within the resulting porous constructs was high (>80%) over 14 days of analysis with multiple cell types. The present invention thus provides a simple, versatile, and tunable method for cell encapsulation within highly porous constructs that can be used for the delivery of cell-based therapies.

The present approach to obtaining cell-laden hydrogel foams allows for direct cell encapsulation within biomaterials without the need for porogens or microcarriers while maintaining high cell viability. The successful encapsulation of multiple cell types into gas-blown hydrogels with varied chemistries shows the versatility of this approach. While the present invention includes photocrosslinkable polymers, any quick gelling materials could be used for foam fabrication. The present invention thus allows for a wide-ranging treatment of diseases and injuries that can benefit from the utilization of cell therapies.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a schematic of cell-encapsulated gas-blown hydrogel foam formation according to the present invention.

FIG. 2 is a diagram of methods for synthesis of poly(vinyl alcohol) methacrylate (PVAMA), poly(ethylene glycol) dimethacrylate (PEGDMA), and gelatin methacrylate (GelMA) used for hydrogel foams.

FIG. 3 is a graph of mesenchymal stem cell viability after 1 hour of exposure to foam blowing components (1 wt % NaHCO₃, 0.2 wt % Irgacure 2959, and 0.25 wt % citric acid) and after 24-hour recovery, measured using a resazurin assay. Horizontal line=75% viability, based on ISO 10993-5 standards for cytocompatibility. N=3, mean±standard deviation displayed

FIG. 4 is a series of graphs of measured pH of solutions containing 1% NaHCO₃ in phosphate buffered saline (DPBS) or Dulbecco's modified Eagle's Medium (DMEM) without (A, B) or with (C, D) 50 mM (HEPES) supplementation. Measurements were taken directly after treatment with 0, 0.25, 0.5, or 0.75% citric acid and again after 1 hour of equilibration. N=3, mean±standard deviation displayed

FIG. 5 is a series of graphs and images of mesenchymal stem cell viability after exposure to buffer solutions containing 1% NaHCO₃ and 50 mM HEPES treated with 0, 0.25, 0.5, and 0.75% citric after 20 minutes and after a 24-hour recovery period. (A) Quantitative cell viability assessed by resazurin assay and (B) visual assessment of cell morphology after straining with live/dead assay. N=3, mean±standard deviation displayed. White scale bar=500 μm and applies to all images. *p<0.05 relative to corollary DPBS solution.

FIG. 6 is a series of scanning electron micrographs of (A) DPBS-based and (B) DMEM-based PVAMA foams with varied citric acid concentrations and varied concentrations of high molecular weight PVA (108 kDa) added as a thickener (PVA-0: 0%, PVA-1: 1%, PVA-2: 2%, PVA-5: 5%). Scale bar=500 μm and applies to all images.

FIG. 7 is (A) a graph of the viscosity profiles of hydrogel solutions and (B) a series of corresponding scanning electron micrographs of hydrogels based on PVA-0L, PEG-3, and GelMA-10 with 0.25, 0.5, and 0.75% citric acid. Scale bar of 500 μm applies to all images. Numbers in top left corner correspond to average pore size.

FIG. 8 is (A) a schematic of hydrogel sample preparation for Live/dead imaging; (B) a series of live/dead images of 3T3 fibroblasts encapsulated in hydrogels with quantified viabilities. n=3, mean±the standard deviation displayed; (C) a series of graphs of 3T3 fibroblast and mesenchymal stem cell (MSC) viability after encapsulation in PVA-0L foams and films (n=3) measured using counts from (D) a series of images after live/dead staining of center cross-sections over 14 days. Scale bar=100 μm. *p<0.05 between film and foam.

### DETAILED DESCRIPTION

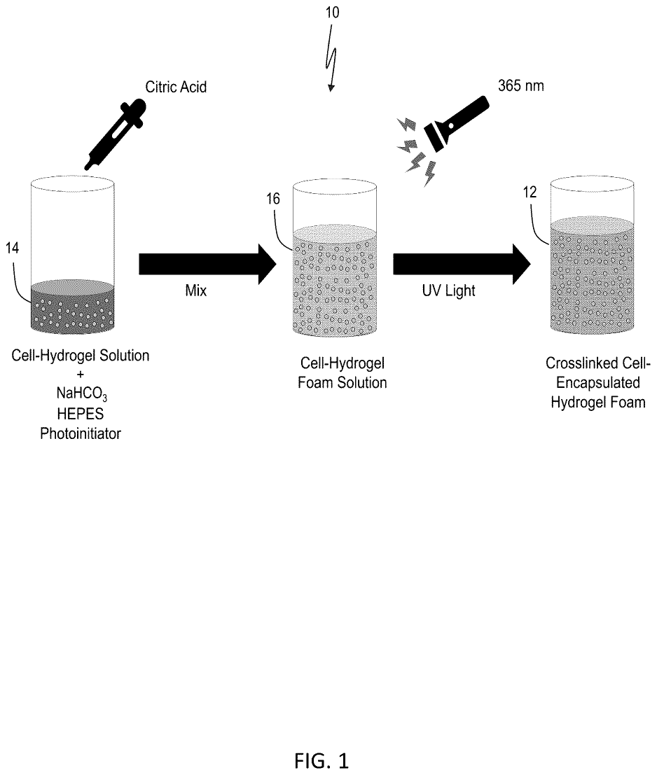

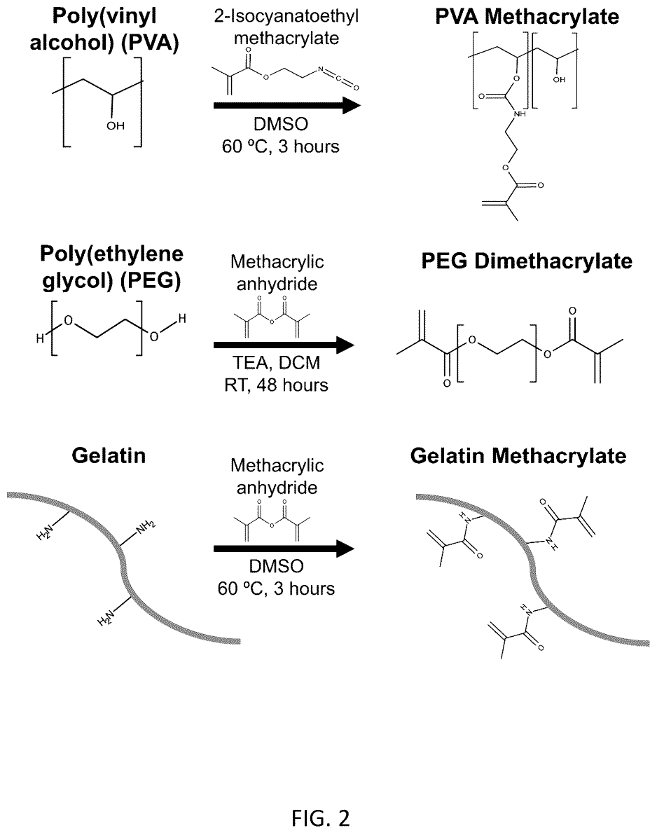

Referring to the figures, wherein like numerals refer to like parts throughout, there is seen in FIG. 1, the present invention provides a method 10 to obtain a cross-linked cell encapsulated hydrogel foam 12. Method 10 commences with a cell-hydrogel solution 14 containing the cells to be encapsulated and a polymer, such as those seen in FIG. 2. Cell-hydrogel solution 14 further includes a cytocompatible blowing agent such sodium hydrogen bicarbonate (NaH₂Co₃), a buffer such as 4-(2-hydroxyethyl)-1-piperazineethanesulfonic acid (HEPES), and a photoinitiator. Citric acid is added to the cell-hydrogel solution 14 and mixed to form a cell-hydrogel foam solution 16. Cell-hydrogel foam solution 16 is then irradiated with UV light to form cross-linked cell encapsulated hydrogel foam 12.

Method 10 thus allows for direct encapsulation within biomaterials without the need for porogens or microcarriers while maintaining high cell viability. The design of the cytocompatible gas foam blowing system of the present invention involves four major components: cytocompatible blowing agents, buffering agents, thickeners, and quick gelling polymers. Improving the cell viability of biological blowing components at higher concentrations by buffering increases the amount of gas that can be produced (and therefore the pores that can be formed) before displaying cytotoxic effects. By controlling the viscosity of these solutions with added thickeners, gas trapping increased at lower concentrations of blowing components, allowing for potential use of these methods with more sensitive cell types in future work. Finally, adjusting the gelling rate of polymers improves the porosity and pore morphology.

The successful encapsulation of multiple cell types into the gas-blown hydrogels of the present invention shows the potential versatility of this method. This system could be used in a range of applications where cell delivery is required. Furthermore, expanding to other therapeutic cell types, such as pancreatic islets for the treatment of type 1 diabetes, could provide new approaches to improving their long-term viability and efficacy. While the present invention was demonstrated using photocrosslinkable polymers, any quick gelling method could be used for foam fabrication in the future expansion of this work, including thermoresponsive materials, Schiff's base, and ionic crosslinking methods. Due to the high tunability of the disclosed process, components like thickeners could be swapped for polymers with desired properties. The impact of this method on the treatment of diseases and injuries that utilize cell therapies is wide-ranging.

### EXAMPLE

### IC50 Values:

3T3 mouse fibroblasts (ATCC) were used to assess initial IC50 values (concentration at which 50% of cells remain alive after exposure) of citric acid, NaHCO₃, and Irgacure 2959 to be used for further solution optimization. Cells were cultured in DMEM with 10% fetal bovine serum (FBS) and 1% penicillin-streptomycin (pen-strep) at 37° C. with 5% CO2. Cells were trypsinized and seeded at a density of 5,000 cells per well in a 96-well plate. Wells (n=3) were then exposed to solutions of citric acid, NaHCO₃, or Irgacure 2959 that were serially diluted in DMEM. After a 20-minute exposure, solutions were removed, and cell viability was measured compared to an untreated control using a resazurin assay (Alamar Blue) read by a plate reader. Cell viability was calculated as:

$$Cell Viability ( \% ) = \frac{{Abs}_{Sample} - {Abs}_{Blank}}{{Abs}_{Control} - {Abs}_{Blank}} \times 100 \%$$

The blank was a well with media only and no cells. Cell viability was plotted for each dilution, and IC50 values were determined as the point where the curves showed 50% cell viability.

### Solution Cytocompatibility:

Using IC50 values as a concentration ceiling, the cytocompatibility of citric acid, NaHCO₃, and Irgacure 2959 was measured with adipose-derived MSCs at concentrations of 0.25, 1, and 0.2% (w/v) in DMEM, respectively. MSCs were seeded in 24 well plates at 2,500 cells per well. Each component (n=3) was added to a well and allowed to incubate for 30 minutes at 37° C. and 5% CO₂. After incubation, cells were treated with a resazurin assay for 2 hours for cytocompatibility measurements or with Live/Dead assay stains and imaging for morphology analysis.

### Buffering:

Buffered solutions containing DPBS or DMEM were supplemented with 1% NaHCO₃ and 50 mM HEPES. To prepare the solutions, 10× concentrated DPBS and DMEM (with NaHCO₃) solutions were made. HEPES (1 M, pH 7) was added to a subset of solutions to provide 50 mM HEPES after diluting to 1×. Then, an additional 1% NaHCO₃ was added. The solutions were equilibrated at room temperature for 1 hour. Then, citric acid was added at 0.25, 0.5, or 0.75%. A DMEM+HEPES control was also tested at 1%. Immediately after adding citric acid, the pH was measured (n=3) using a pH probe (Thermo Scientific). Solutions were incubated at 37° C. and 5% CO2 for 1 hour, and then pH was retested.

### Buffered Solution Cytocompatibility:

Solutions containing DMEM or DPBS were supplemented with 50 mM HEPES, 1% NaHCO₃, and 0.2% Irgacure 2959. Then, solutions were filtered through a 0.20 μm syringe filter. MSCs were seeded into 24 well plates at 2,500 cells per well the night before testing. Buffered solutions were added (500 μL) to each well. Citric acid was then added to the wells at 0.25, 0.5, or 0.75% (n=3). The plate was placed into a UV curing box (Analytik Jena CL-1000) with a wavelength of 365 nm for 5 minutes. The plate was removed and incubated at 37° C. and 5% CO2 for 20 additional minutes. The samples were then stained with a resazurin assay for cell viability measurement or with a Live/Dead assay for images and morphology assessments. After testing, fresh DMEM supplemented with 10% FBS and 1% pen-strep was added to each well and incubated for an additional 24 hours. Live/dead and resazurin assays were completed again to evaluate cell recovery.

### Polymer Synthesis:

PVA was methacrylated using 2-isocyanatoethyl methacrylate (ICEMA) in DMSO, FIG. 2. Briefly, vacuum dried PVA (25 kDa) was dissolved in DMSO at 96° C. to make a 10% solution under nitrogen. The solution was cooled to 60° C., and ICEMA was added dropwise at a 4% molar ratio of isocyanates to PVA hydroxyls. The reaction proceeded for 3 hours at 60° C., and the product was precipitated in cold acetone, dried, redissolved in water, and then reprecipitated in cold acetone. The final product was vacuum dried for 24 hours. Nuclear magnetic resonance (NMR) spectroscopy confirmed methacrylation of PVA by comparing the PVA backbone (δH 3.7-4.1 and 1.4-1.8 p.p.m.) and the methacrylate vinyl protons (δH 5.6 and 6.1 p.p.m). PVAMA had a degree of substitution of backbone pendant hydroxyls of 3%.

PEGDMA was synthesized using methacrylic anhydride (MA) with triethylamine (TEA) as a catalyst, FIG. 2. PEG (4 kDa) was dissolved in dichloromethane at 33 wt % with a small amount of hydroquinone. MA and TEA were added at a molar ratio of 2.2 MA:2 TEA:1 PEG. The reaction proceeded at room temperature under nitrogen for 3 days. The polymer was then precipitated in cold ether and vacuum dried. Methacrylation was characterized using NMR spectroscopy by comparing the adjacent proton to the methacrylate group (δH 4.3 p.p.m.) and the methacrylate vinyl protons (δH 5.6 and 6.1 p.p.m). PEGDMA had a degree of substitution of terminal hydroxyls of 97%.

GelMA was synthesized using MA, FIG. 2. Gelatin was dissolved in DMSO at 60° C. Methacrylic anhydride was then added to the solution at 0.1 g/g of gelatin. The reaction proceeded for 3 hours at 60° C. The polymer was precipitated in cold ethanol, redissolved in water, and reprecipitated in ethanol. The final product was vacuum dried and characterized via Fourier transform infrared spectroscopy.

### Rheology

Rheological measurements were performed using a TA Instruments Discovery Rheometer with a 40 mm plate geometry. Viscosity measurements were taken with a goal of matching viscosities that of PVA-0L (Table 1), which produced highly porous foams. Viscosities of PEGDMA and GelMA hydrogel solutions were tuned with thickeners of poly(ethylene oxide) (PEO, 400,000 Da) and cornstarch, respectively. Tested samples included PVA-0L, PEG-0, PEG-3, and GelM-10, Table 1. Solutions were conditioned at 37° C. for 120 seconds. The samples were then tested using a flow sweep at shear rates between 1-100 radians per second.

### Hydrogel Foam Fabrication

Hydrogel solutions were prepared by dissolving 1 g of PVAMA and 0.1 g of gelatin (surfactant) with or without PVA (108k) thickeners in a solution containing 4.25 ml of DI water and 250 μL of 1 M HEPES at 96° C. The solution was cooled to below 50° C., and 500 μL of 10×DMEM or 10×DPBS was added. NaHCO₃ and Irgacure 2959 were then added so that the final concentrations were 20% PVAMA, 2% gelatin, 1% NaHCO₃, 0.2% Irgacure 2959, and 50 mM HEPES. Thickeners were added at 0, 1, 2, and 5%, Table 1. The effect of the photoinitiator on pore morphology was also characterized by replacing Irgacure 2959 with LAP at a concentration of 0.05% (w/v). Only hydrogels containing DMEM were examined for use with LAP, Table 1. PEGDMA and GelMA hydrogels were formulated using the same method, using surfactant and thickeners shown in Table 1. PEGDMA hydrogel solutions were all dissolved at room temperature while GelMA was dissolved at 90° C.

To form hydrogels, 1 ml of solution was placed into a 24-well plate. Citric acid was then added from a 5% stock solution to a final concentration of 0.25, 0.5, or 0.75% (50, 100, or 150 μL, respectively). The plate was then vigorously shaken on a vortex mixer for 20 seconds and quickly transferred into a UV curing box. Solutions were irradiated for 5 minutes (Irgacure 2959) or 2 minutes (LAP). Samples were air dried, cut down the center, sputter coated with gold, and imaged using SEM (Jeol NeoScope JCM-5000) at 35× magnification and 10 kV to qualitatively examine pore structures. In samples with visible porosity, pore size was examined by measuring 15 random pores per image (n=30 pores) using the measurement tool in ImageJ.

TABLE 1
Synthesized hydrogel formulation names and solution compositions. PVA: Poly(vinyl alcohol);
PEG: Poly(ethylene glycol); Gel: Gelatin; MA: methacrylate; I2959: Irgacure 2959;
DMEM: Dulbecco's modified Eagle's medium; DPBS: Phosphate buffered saline.

| | | | | | | | Citric | |
|---|---|---|---|---|---|---|---|---|
| | | | | | NaHCO₃ | HEPES | Acid | Media |
| Name | Polymer | Surfactant | Initiator | Thickener | (% w/v) | (mM) | (% w/v) | Type |
| PVA-0 | 20% | 2% Gel | I2959 | N/A | 1 | 50 | 0.25, 0.5, | DMEM, |
| | PVAMA | | (0.2%) | | | | 0.75 | DPBS |
| PVA-1 | 20% | 2% Gel | I2959 | 1% PVA | 1 | 50 | 0.25, 0.5, | DMEM, |
| | PVAMA | | (0.2%) | 108k | | | 0.75 | DPBS |
| PVA-2 | 20% | 2% Gel | I2959 | 2% PVA | 1 | 50 | 0.25, 0.5, | DMEM, |
| | PVAMA | | (0.2%) | 108k | | | 0.75 | DPBS |
| PVA-3 | 20% | 2% Gel | I2959 | 5% PVA | 1 | 50 | 0.25, 0.5, | DMEM, |
| | PVAMA | | (0.2%) | 108k | | | 0.75 | DPBS |
| PVA-0L | 20% | 2% Gel | LAP | N/A | 1 | 50 | 0.25, 0.5, | DMEM |
| | PVAMA | | (0.05%) | | | | 0.75 | |
| PEG-0 | 20% | 2% F-127 | LAP | N/A | 1 | 50 | N/A | DMEM |
| | PEGDMA | | (0.05%) | | | | | |
| PEG-3 | 20% | 2% F-127 | LAP | 3% PEG | 1 | 50 | 0.25, 0.5, | DMEM |
| | PEGDMA | | (0.05%) | 400K | | | 0.75 | |
| Gel-10 | 10% | N/A | LAP | 10% | 1 | 50 | 0.25, 0.5, | DMEM |
| | GelMA | | (0.05%) | Starch | | | 0.75 | |

### Cell Encapsulation

PVA-0L, PEG-3, and Gel-10 solutions were tested with 3T3s for cell encapsulation. PVA-0L was also used to encapsulate MSCs. Polymer solutions were sterile filtered, and then cells were added at 5×10⁴ cells/ml. Polymer-cell suspensions (500 μL) were placed into a 24 well plate (n=3). Filter-sterilized 5% citric acid (75 μL) was added to each well, and the plate was vortexed for 30 seconds and then placed under UV light for 2 minutes. Upon completion of crosslinking, 1 mL of cell culture media was placed in each foam containing well for 20 minutes. The media was then changed, and cells were allowed to incubate at 37° C. for 24 hours before live/dead staining. Cells were encapsulated in control samples without citric acid added to form non-porous hydrogels. At selected time points, samples were cut into 3 lateral cross-sections and placed in Live/Dead assay stain solution for 30 minutes. The samples were washed with PBS and then imaged. Cell viability was calculated by:

$$Cell Viability ( \% ) = \frac{Live Cells}{Total Cells} \times 100 \%$$

### Statistical Analysis:

Measurements are presented as mean standard deviation. Single factor ANOVA with Tukey's post hoc were used determine significance between groups. A p-value of <0.05 was taken as statistically significant.

### Experimental Results

### IC50 Values and Solution Cytocompatibility

TABLE 2
IC50 values for foaming components based
on testing with 3T3 mouse fibroblasts.

| Component | IC50 (% w/v) |
|---|---|
| Citric acid | 0.23 |
| Irgacure | 0.80 |
| NaHCO₃ | 1.5 |

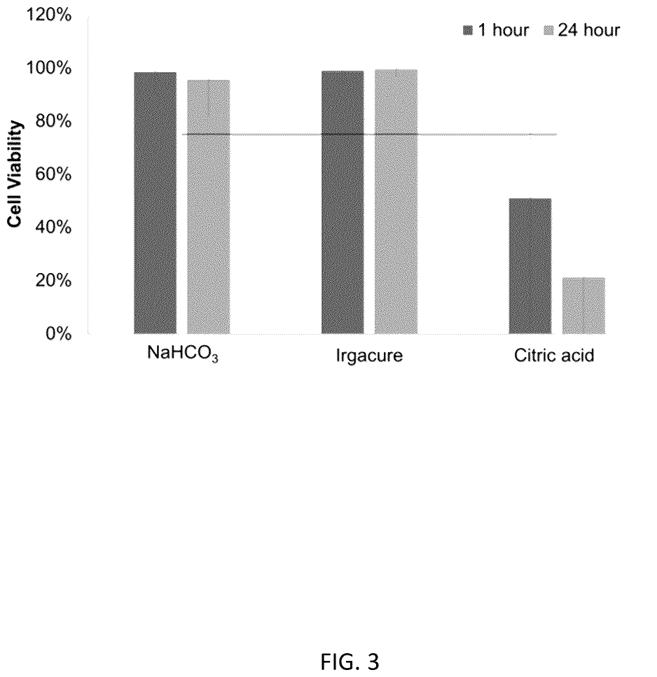

The IC50 values for the critical foam blowing components, citric acid, Irgacure 2959, and NaHCO₃, were 0.23, 0.80, and 1.5%, respectively, Table 2. Using this data, the cytocompatibility of the critical blowing components (citric acid, Irgacure 2959, and NaHCO₃) at concentrations of 0.25, 0.2, and 1% (w/v, respectively) was examined with MSCs, FIG. 3. Both NaHCO₃ and Irgacure 2959 showed cytocompatibility above the 75% benchmark with values of 98 and 99% viability after 1 hour of exposure and 95 and 99% after the 24-hour recovery, respectively. Citric acid exposure resulted in reduced cell viability (51% and 21%) after both the 1-hour treatment period and the 24-hour recovery period, respectively.

### Buffering Capacity

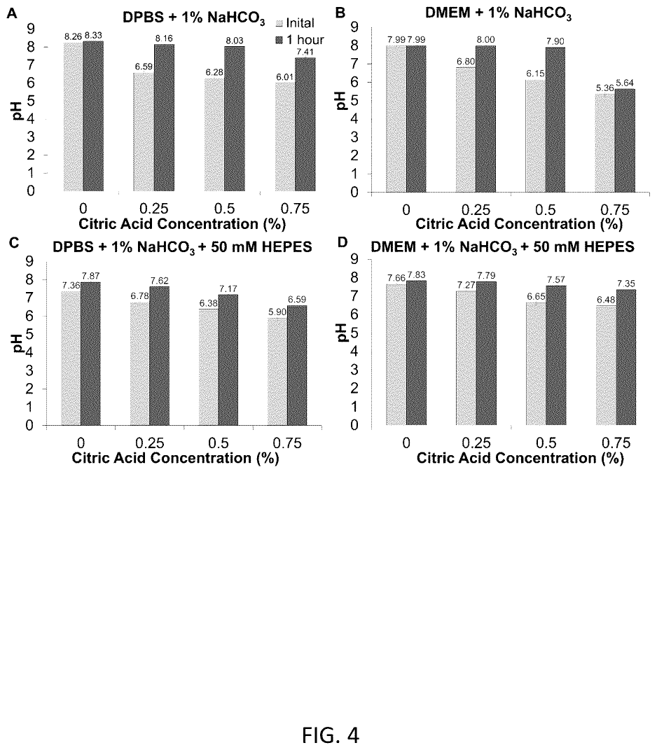

The DPBS solution containing 1% NaHCO₃ showed pH values of 8.33, 8.16, 8.03, and 7.41 after 1 hour of treatment with 0, 0.25, 0.5, and 0.75% citric acid, FIG. 4A. DMEM with 1% NaHCO₃ had pH values of 7.99, 8.00, 7.90, and 5.64 after 1 hour of incubation with increasing concentrations of citric acid, FIG. 4B. When supplemented with HEPES, the pH of the DPBS solutions at 1 hour (7.87, 7.62, 7.17, and 6.59) was lower than the corollary non-buffered DPBS solutions, FIG. 4C. Similar trends were seen in HEPES-buffered DMEM solutions, except that pH was increased in HEPES containing DMEM with 0.75% citric acid. The pH values of the DMEM+HEPES solutions were 7.83, 7.79, 7.57, 7.35, and 7.05, FIG. 4D. In general, DPBS solutions had higher pH compared to corollary DMEM solutions with and without HEPES.

### HEPES Solution Cell Viability

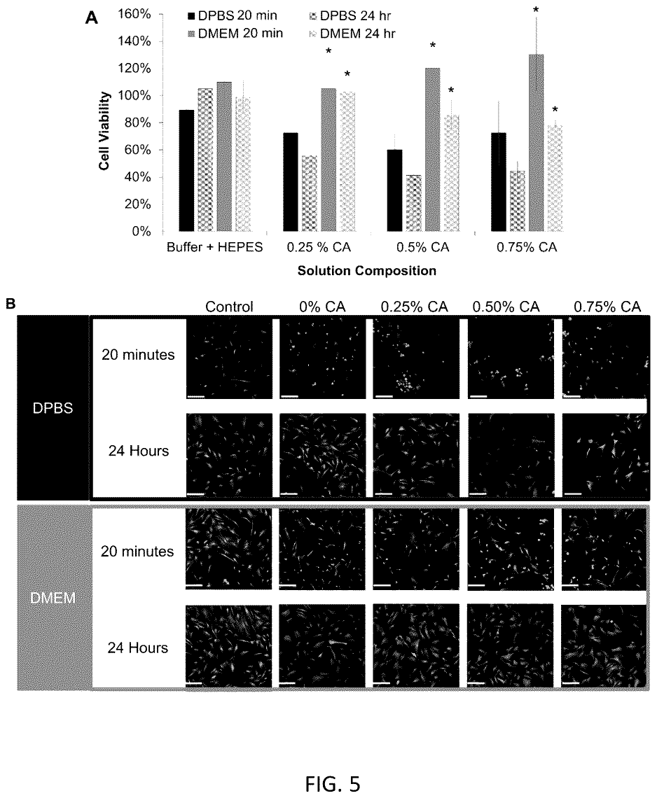

All tested solutions had MSC viability above 75% before citric acid treatment initially and after 24 hours of recovery, FIG. 5A. DPBS solutions supplemented with citric acid all showed cell viability <75%, with further decreased in viability at 24 hours. DMEM solutions had high cell viability (>100%) after initial treatment with all concentrations of citric acid and >75% viability after the 24-hour recovery, with 0.75% citric acid having the lowest MSC viability at 78%.

Cells treated with DPBS solutions underwent morphological changes, even when viability measurements were high (0% citric acid), FIG. 5B. In general, cell rounding increased with increased citric acid. Fewer morphological changes were observed in DMEM-based solutions. MSCs appeared smaller in all testing solutions when compared to the DMEM control directly after exposure. Morphology returned to normal after the 24-hour recovery in both DMEM and DPBS solutions, although DPBS wells had qualitatively lower cell density than DMEM samples after recovery.

### Irgacure 2959-Initiated Hydrogel Foam Morphology

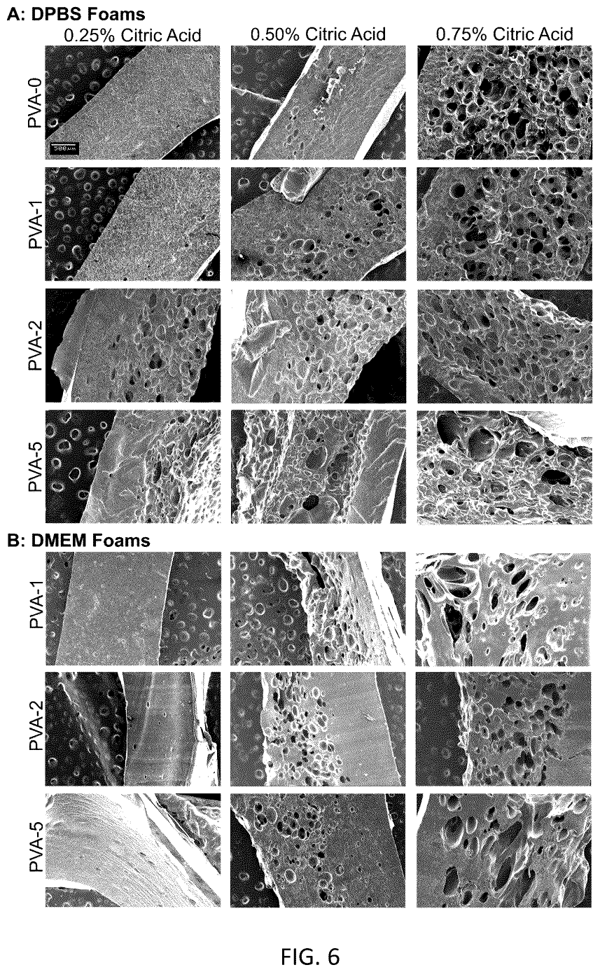

PVAMA-based hydrogels showed improved porous structure with increasing concentrations of both high molecular weight PVA (thickener) and citric acid. Hydrogels formed with DPBS were able to form pores without the use of a thickener (PVA-0), FIG. 6A. DMEM-based PVA solutions crosslinked with Irgacure were unable to form porous hydrogels without added thickener (images not included). While general trends of improved pores with increased thickener and citric acid were maintained in foams formed in DMEM, these scaffolds showed overall lower visible porosity than foams fabricated in DPBS, FIG. 6B.

### LAP-Initiated Hydrogel Foam Morphology

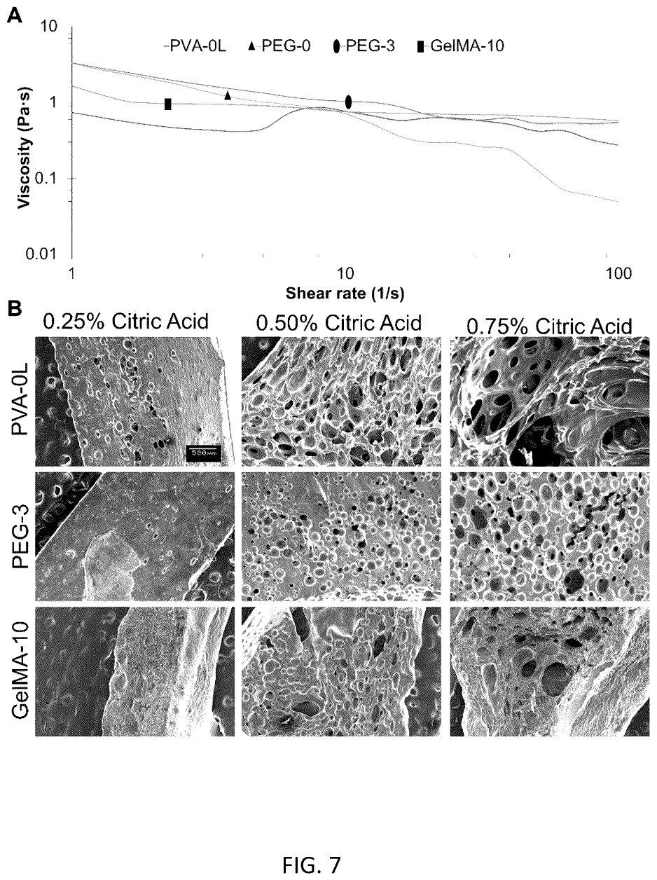

Based on its faster initiation rates, LAP was used in place of Irgacure 2959 to study the ability to use these foaming methods with multiple polymer types. First viscosity was measured, based on the observations that thickeners qualitatively affected both viscosity and foaming (better pore structures with increased thickener) in initial studies, FIG. 7A. At higher shear rates (100 radians/seconds), PEG-0 solutions (yellow line) showed the lowest viscosity of 0.05 Pa s followed by PVA-0L (blue line) with a viscosity of 0.27 Pa s. When thickeners were added to GelMA-10 (grey line) and PEG-3 (orange line) solutions, the viscosity was increased to 0.58 and 0.54 Pa s, respectively, at 100 radians/second. PEG-0 exhibited the most shear-thinning, with reduced viscosity as shear rate increased.

These solutions were utilized to form hydrogel foams with the addition of varying concentrations of citric acid, FIG. 7B. PEG-0 hydrogels were unable to form foams and were not imaged. Low porosity was observed with 0.25% citric acid, and increased porosity was seen with increased citric acid content. The PVA-0L hydrogels showed highly porous structures at both 0.5 and 0.75% citric acid, and better pore structures were formed than those observed in the Irgacure-based PVA hydrogels shown in FIG. 6. PEG-3 and GelMA-10 hydrogels also showed increasing porosity with increased citric acid, with more regular pores observed in PEG-3 hydrogels.

### Cell Encapsulation

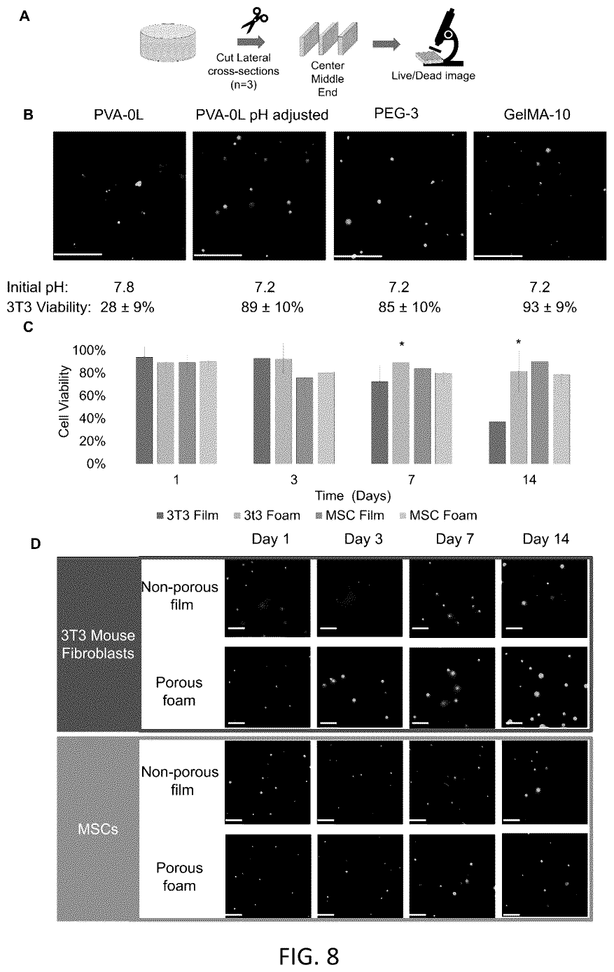

3T3 fibroblast encapsulation was initially assessed with PVA-0L, PEG-3, and GelMA-10 hydrogels. Cells encapsulated inside of PVA-0L with 75% citric acid showed low cell viability (28±9%), FIG. 8B. Adjustment of the initial pH to 7.2 by adding 1 M HCl to PVA0-L hydrogel solutions significantly improved cytocompatibility to 89±10%. Encapsulated 3T3s had high cytocompatibility in PEG3 and GelMA-10 hydrogels formed with 0.75% citric acid (85%).

Using this information on the importance of initial pH, 3T3 and MSC viability was further assessed over up to 14 days in PVA-0L films and foams. There were no significant differences in viability between 3T3s or MSCs encapsulated in films or foams up to day 3, FIGS. 8C and D. On day 7, foams showed higher 3T3 viability in comparison to films, while MSC viability was comparable between the films and foams. Further declines in 3T3 viability were seen in films at 14 days, with high viability maintained in porous foams. MSCs showed no significant differences in cytocompatibility between the film and foam at any timepoint.

### DISCUSSION

Since 50-70% of encapsulated cells were not viable in previous work to obtain gas-blown hydrogel foams, obtaining IC50 values of critical blowing components (citric acid, Irgacure 2959, and NaHCO₃) and using that information to improve viability was important to increasing viability of encapsulated cells. The calculated IC50 values for citric acid and NaHCO₃ were 5-10× lower than the concentrations used in previous work. The IC50 value of Irgacure 2959 was 0.8%, which is well above concentrations that are typically used for cell encapsulation in photocurable polymers. Based on these studies, 1 wt % NaHCO₃, 0.2 wt % Irgacure 2959, and 0.25 wt % citric acid were selected as initial starting values for foam development. Citric acid was used at a concentration that is slightly higher than the IC50 value of 0.23 wt %, as it is the component that is required for the production of CO₂ to form porous foams during crosslinking.

While 3T3s are good model cells for initial characterization, MSCs were employed to assess cytotoxicity with a therapeutic cell type. Both Irgacure 2959 and NaHCO₃ had MSC cytocompatibility above the benchmark of 75% set by ISO 10993-5. Citric acid was the only major blowing component with observed toxic effects on MSCs. Citric acid has previously shown to limit cell growth and attachment, but most prior studies do not adjust the pH when introducing citric acid. To that end, the effect of gaining tighter control over the solution pH was examined by buffering. Buffers commonly used in cell culture are NaHCO₃ and HEPES. The current foaming solution already employs close to cytotoxic concentrations of NaHCO₃, so HEPES, a zwitterion, was used to supplement the blowing solutions and improve pH control. The pH of foam solutions in DPBS and DMEM containing 1% NaHCO₃ with or without 50 mM HEPES were measured to test this hypothesis.

In general, DPBS-based solutions containing only 1% NaHCO₃ had a higher pH than corollary solutions in DMEM. Immediately after the addition of citric acid, the solution pH decreased, and then it gradually increased. Higher concentrations of citric acid generally reduced pH values. HEPES-supplemented solutions had smaller differences between initial and equilibrated pH values in comparison to NaHCO₃ alone, except for 0.75% citric acid solution in DMEM. HEPES supplementation increased the initial pH in comparison to NaHCO₃ alone except for 0.75% citric acid in DPBS. pH values above 7.8 or below 6.8 can cause cell necrosis and apoptosis. Solutions supplemented with only NaHCO₃ without the addition of citric acid had pH values outside of this range (7.99: DMEM; 8.33: DPBS), demonstrating that the addition of citric acid is important for neutralizing the added NaHCO₃. As an extra step, pH values of DMEM solutions containing HEPES with 1% citric acid were measured. This solution had an initial pH of 6.07, which is lower than the ideal range for cell culture. However, the equilibrated 1-hour pH was increased to a physiologically relevant level of 7.05. In general, this investigation shows that HEPES increases the buffering capacity of the solutions to make them more resistant to drastic pH changes, which could increase cell viability while allowing for use of higher concentrations of citric acid to improve bubble formation and gas blowing.

Once it was understood how to control foaming solution pH, the cytocompatibility of buffered foam solutions with MSCs was characterized. Improved MSC viability was observed in the presence of 0.25% citric acid in buffered solutions as compared with initial IC50 value testing with 3T3s. At 24 hours, DPBS solutions showed 2× higher MSC viability (vs. 3T3 viability), but viability was still below the 75% benchmark. DMEM solutions had cell viability >75% for citric acid concentrations up to 0.75%. Initial viability after the addition of citric acid was consistently higher than 24-hour viability, which is attributed to citric acid's role in the Krebs cycle; namely, the resazurin assay used to measure cell viability is a metabolic assay, and cell metabolism is increased in the presence of citric acid.

To qualitatively confirm initial cell viabilities and to evaluate effects of foaming solutions on cell morphology, live/dead images were taken at 20 minutes and 24 hours after treatment. After 20 minutes, all solutions with or without added citric acid showed cell shrinking. This shrinking is attributed to the hypertonic concentration of NaHCO₃, causing cells to lose water to equilibrate the surrounding solution. Cells exposed to DPBS-based solutions showed increased cell rounding, detachment, and death compared to those in corollary DMEM solutions. After 24 hours of recovery, cells regained size as compared to the control solution. Overall, using DMEM-based foam solutions resulted in better cellular characteristics for use in the development of gas-blown foams with encapsulated cells.

Using this information, the effects of polymer type, citric acid content, and viscosity on foam porosities was characterized to determine whether porous foams could be fabricated using cytocompatible foaming solutions. Initial attempts to fabricate foams using the PVA-0 formulation only showed successful pore formation at a concentration of 0.75% citric acid in DPBS. No foams were formed using DMEM and the PVA-0 formulation. Due to the poor cell viability of the DPBS solution with 0.75% citric acid, the solution's rheological properties were tuned to improve foaming outcomes. Thickeners are often used to increase the viscosity of gas-blown foam solutions and tune porosity. Thus, it was hypothesized that adding thickeners in the form of high molecular weight polymers could be used to achieve higher porosities at lower concentrations of citric acid.

The PVA-1 formulation with 1% PVA (108 kDa) formed porous structures at a concentration of 0.5% citric acid in both DMEM and DPBS-based solutions. In PVA-2 and PVA-5 formulations with 2 and 5% PVA (108 kDa) in DPBS, porous structures begin to form at even lower concentrations of citric acid (0.25%). DMEM solutions with 0.25% citric acid showed limited pore formation, which is attributed to the higher buffering compacity of DMEM. Namely, at a citric acid concentration of 0.25%, the solutions do not reach the acidic pH required for the release of CO₂ by NaHCO₃. While adding thickeners to increase solution viscosity improved pores, visible pore content in DMEM-based foams was inferior to that in DPBS foams. The pore morphology of 0.75% citric acid foams in DMEM show more elongated and larger pores, which can be caused by slow gelling or crosslinking. Hydrogel crosslinking may be slower in DMEM than that in DPBS due to interactions with DMEM components, such as antioxidants like phenol red. It was hypothesized that it was possible to increase the speed of gelling and better trap CO₂ air bubbles in DMEM-based foams by using a more efficient photoinitiator.

Many studies have shown LAP to have more efficient initiation compared to Irgacure 2959, even at lower concentrations. Another benefit of using LAP is that it can be initiated using more cell-friendly visible light, which could be harnessed in future work to eliminate the need for UV light. Due to the more efficient formation of free radicals by LAP, extended curing times can increase cytotoxicity; thus, irradiation times were decreased to 2 minutes in LAP-initiated hydrogels. Substitution of Irgacure 2959 with LAP resulted in drastic improvements to pore structure, even at lower initiator concentrations and shorter curing times. The PVA-0L foam formed with LAP was a highly porous scaffold at citric acid concentrations of 0.5 and 0.75%, without the need for thickeners. This result shows the importance of balancing both gelling and blowing processes to achieve ideal foam properties. These hydrogels also showed interconnected pore structures, which is ideal for implantable hydrogels to allow for tissue and vascular ingrowth.

While this system was initially designed using PVAMA, the concepts are translatable to other polymers. To demonstrate the adaptability of this approach, PEGDMA and GelMA were employed to fabricate porous hydrogels. Initially, the PEG-0 formulation was utilized, but this composition did not yield a porous foam. Thus, it was an aim to increase the viscosity of the PEGDA solution to values that are similar to or slightly higher than that of PVA-0L. Using the PEG-3 solution with 3% PEO (400 kDa) added as a thickener, the viscosity was increased to obtain porous hydrogels. The same principle was applied to the GelMA-10 formulation, wherein a natural, biodegradable thickener, cornstarch, was added at 10% to provide highly porous hydrogel foams. Utilizing a completely natural polymer system for cell encapsulation could allow for cellular remodeling of the construct during healing. It should be noted that the addition of thickeners has the potential to affect mechanical properties, but corn starch could be quickly removed by treatment with the cytocompatible enzyme, amylase. While all three polymers successfully formed foams, pore morphology differed between the materials. Other factors, such as surfactant type, concentration, and polymer interactions can impact foaming. Future studies could examine how adjusting these parameters can be used to optimize foam structures for selected polymers and applications.

Initially, cell encapsulation was completed using 3T3 cells as a proof-of-concept for the adaptability of this system. Encapsulated 3T3s had low viability in the PVA-0L hydrogel foams, but PEG-3 and GelMA-10 showed low toxicity 24 hours after encapsulation. The PEG-3 and GelMA-10 solutions had initial pH of 7.2, while PVA-0L had an initial pH of 7.8. Thus, pH of the PVA-0L solution was adjusted to 7.2, and 3T3 encapsulation was repeated to result in significantly increased cell viability. Further study on the specific pH range required to achieve high cell viability is needed in future work, but a pH of 7.2 was utilized in the remaining studies.

Both 3T3s and MSCs were then encapsulated in non-porous hydrogel films and porous hydrogel foams (PVA-0L) and characterized over 2 weeks to assess long-term viability. Both cell types had similarly high viabilities after 24 hours, and at 7 days of culture, foams and films showed no statistical differences in cell viability. However, continued culture of 3T3s for 14 days showed a large decline in viability in the films, while foams maintained cytocompatibility over 80%. This result confirms previous work stating that encapsulation within hydrogels without adequate channels for nutrient waste transport results in decreased viability over extended culture times. MSCs showed no significant differences in cell viability in foam and films, but were successfully encapsulated within a PVA-0L with viability of 89%. It should be noted that the same volume of hydrogel solution was used for hydrogel film and foam synthesis, resulting in thinner film scaffold relative to foams. Differences between cell viabilities over time would therefore be more likely to be pronounced in thicker hydrogel films.

What is claimed is:

1. A cell encapsulated hydrogel foam system, comprising
an amount of cells to be encapsulated;
a gelating polymer;
an amount of a buffer; and
a cytocompatible foaming agent.

2. The system of claim 1, wherein the cytocompatible foaming agent comprises an amount of sodium bicarbonate and an amount of citric acid.

3. The system of claim 1, the gelating polymer is methacrylated and selected from the group consisting of poly(vinyl alcohol), poly(ethylene glycol), and gelatin.

4. The system of claim 3, wherein the amount of the buffer will achieve a pH of 7.2.

5. The system of claim 1, further comprising an amount of a photoinitiator.

6. The system of claim 1, further comprising an amount of a thickener.

7. A method of encapsulating cells, comprising the steps of:
providing a hydrogel formed from a gelating polymer;
suspending an amount of cells to be encapsulated in the hydrogel;
adding an amount of a cytocompatible buffer;
adding an amount of a cytocompatible foaming agent to the hydrogel;
agitating the hydrogel to form a hydrogel foam; and
cross-linking hydrogel foam to form a cross-linked cell encapsulated hydrogel foam.

8. The method of claim 7, wherein the gelating polymer is methacrylated and is selected from consisting of poly(vinyl alcohol), poly(ethylene glycol), and gelatin.

9. The method of claim 7, wherein the amount of the cytocompatible foaming agent comprises an amount of sodium bicarbonate and an amount of citric acid.

10. The method of claim 7, wherein the cytocompatible buffer is 4-(2-hydroxyethyl)-1-piperazineethanesulfonic acid.

11. The method of claim 10, wherein the amount of the buffer achieves a pH of 7.2.

12. The method of claim 7, further comprising the step of adding a thickener.

13. The method of claim 7, further comprising the step of adding a photoinitiator.

14. The method of claim 13, wherein the step of cross-linking the hydrogel foam to form the cross-linked cell encapsulated hydrogel foam comprises the step of irradiating the hydrogel foam with ultraviolet light to form the cross-linked cell encapsulated hydrogel foam.

15. An encapsulated cell system, comprising an amount of cells encapsulated in a cross-linked hydrogel foam, wherein the cross-linked hydrogel foam is formed from a methylated gelating polymer and characterized by a pH of 7.2 and a cell viability of at least 90 percent.
